(19) United States
(12) Patent Application Publication
(10) Pub. No.: US 2006/0100530 A1
(43) Pub. Date: May 11, 2006

(54) Systems and methods for non-invasive detection and monitoring of cardiac and blood parameters

(75) Inventors: Michel Kliot, Bellevue, WA (US); Robert C.A. Frederickson, Victoria (CA); Kamran Forghani, Victoria (CA); Pierre D. Mourad, Seattle, WA (US)

Correspondence Address: SPECKMAN LAW GROUP PLLC, 1201 THIRD AVENUE, SUITE 330, SEATTLE, WA, 98101, US

(73) Assignees: Allez PhysiOnix Limited, Seattle, WA (US); University of Washington, Seattle, WA (US)

(21) Appl. No.: 11/234,914

(22) Filed: Sep. 26, 2005

Related U.S. Application Data
(63) Continuation-in-part of application No. 10/861,197, filed on Jun. 3, 2004.
(63) Continuation-in-part of application No. 09/995,897, filed on Nov. 28, 2001, now Pat. No. 6,875,176.
(60) Provisional application No. 60/613,045, filed on Sep. 24, 2004.
(60) Provisional application No. 60/508,836, filed on Oct. 1, 2003.
(60) Provisional application No. 60/475,803, filed on Jun. 3, 2003.
(60) Provisional application No. 60/253,959, filed on Nov. 28, 2000.

(51) Int. Cl. A61B 5/02

(52) U.S. Cl. 600/483

(57) ABSTRACT

Methods and systems for long term monitoring of one or more physiological parameters such as respiration, heart rate, body temperature, electrical heart activity, blood oxygenation, blood flow velocity, blood pressure, intracranial pressure, the presence of emboli in the blood stream and electrical brain activity are provided. Data is acquired non-invasively using ambulatory data acquisition techniques.

### REFERENCE TO PRIORITY APPLICATIONS

This application claims priority to U.S. Provisional Application No. 60/613,045 filed Sep. 24, 2004. This application is also a continuation-in-part of U.S. patent application Ser. No. 10/861,197, filed Jun. 3, 2004, which claims priority to U.S. Provisional Application No. 60/475,803 filed Jun. 3, 2003 and U.S. Provisional Application No. 60/508,836, filed Oct. 1, 2003 and is a continuation-in-part of U.S. patent application Ser. No. 09/995,897, filed Nov. 28, 2001, issued as U.S. Pat. No. 6,875,176 on Apr. 4, 2005, which claims priority to U.S. Provisional Application No. 60/253,959, filed Nov. 28, 2000. These patent applications are incorporated herein by reference in their entireties.

### TECHNICAL FIELD OF THE INVENTION

In one aspect, the present invention relates to methods and systems for monitoring physiological parameters such as respiration, cardiac and/or vascular parameters, events and anomalies, such as embolic events, on an intermittent or continuous basis, using systems that are portable and ambulatory, over an extended period of time. Blood flow parameters, events and anomalies are monitored and detected using non-invasive ultrasound techniques. Cardiac parameters, events and anomalies are monitored, for example, using non-invasive pressure-sensing and ECG technologies. Ambulatory monitoring systems incorporate data recording, processing and storage capabilities for recording and/or storing acquired data, optionally processing acquired data to determine and output one or more physiological parameters, uploading and downloading data and/or instruction sets, inputting patient data, and triggering one or more alarms or notifications. Data analysis may be performed by the ambulatory device and/or by a companion analytical system to which data is uploaded.

### BACKGROUND OF THE INVENTION

Systems for monitoring numerous physiological parameters are well known and are used widely in health care settings. These systems provide a generally high level of data collection and analysis but few of these systems are ambulatory and few provide long term monitoring and data analysis over a period of several days, months or years. Yet, many physiological irregularities manifest only periodically or may be asymptomatic and are difficult to detect during routine patient evaluation, for example, during an appointment with a health care professional or during a hospital stay. Ambulatory heart rate monitors are available commercially and are used for fitness training, cardiac rehabilitation and the like. Some data storage and analytical features are provided, alarms may be programmed or programmable, and various levels of information may be displayed. These systems generally don't have the capability and aren't intended to provide recording and storage of heart rate data for an extended time period. Heart rate monitors typically use a chest band having one or more electrodes to detect heart rate, although monitoring at sites other than the chest using other modalities can be done.

For patients having cardiac irregularities or symptoms that occur sporadically or are asymptomatic, cardiac ECG monitoring is performed over a period of time using portable, battery-operated Holter monitoring or cardiac event monitoring devices and techniques. Holter monitoring is a common type of ambulatory ECG monitoring in which the electrical cardiac signals are detected by electrodes contacting the chest and connected to a recording device. A patient typically keeps a detailed diary of activities and symptoms for a 24 or 48 hour period, during which time the cardiac monitoring takes place so that irregularities are detected and associated with patient activities and symptoms. Holter monitoring is used to identify cardiac arrhythmias as well as transient ischemic episodes and silent myocardial ischemia.

Holter monitors generally record every heartbeat for a recording period, providing continuous cardiac ECG data over the recording period and are typically worn for 24 to 48 hours. Presymptom (looping memory) cardiac event monitors constantly monitor and provide short-term recording of ECG signals. When symptoms occur, the patient presses a button that makes a permanent recording of the ECG data both prior to and following activation of the button. Patient-activated looping memory monitors are typically worn for 30 days, but only patient-initiated events are permanently recorded. A postsymptom event monitor is generally used only when symptoms of a heart problem occur. The patient activates the system to start an ECG recording following the onset of symptoms. Recorded Holter and event monitor data are generally analyzed off-line using dedicated diagnostics systems and services. Programmable, auto-trigger monitors are available for arrhythmia detection. Such devices have been found to be particularly useful for monitoring events that are asymptomatic, such as asymptomatic arrhythmias, Tachycardia, Bradycardia and Pauses.

Although Holter and cardiac event monitors are being used in attempts to diagnose and monitor various cardiac irregularities that are asymptomatic or infrequently experienced, their limited data storage and analysis capabilities have reduced their application for wider ranging diagnostic and monitoring applications. The success rate is rather low with these devices, since the Holter monitor seldom captures rare events in the typical, relatively short-term recording period and event monitor is patient-triggered and user dependent. These systems could be improved with more substantial recording and data storage capability and better analytical systems. The Holter and cardiac event monitors also are typically operated as stand-alone devices and are not interfaced with other devices collecting clinically useful patient data. Nonetheless, Holter and cardiac event monitoring are the only longer-term cardiac event monitoring systems presently available.

Doppler ultrasound techniques measure the frequency shift (the “Doppler Effect”) of reflected sound, which indicates the velocity of the reflecting material. Long-standing applications of Doppler ultrasound include monitoring of the fetal heart rate during labor and delivery and evaluating blood flow in the carotid artery. The use of Doppler ultrasound has expanded greatly in the past two decades, and Doppler ultrasound is now used in many medical specialties, including cardiology, neurology, radiology, obstetrics, pediatrics, and surgery. Transcranial Doppler (TCD) technology today allows detection of blood flow in intracranial arteries and is used for interoperative monitoring, to detect intracranial stenoses, to measure dynamic cerebrovascular responses, and to detect emboli.

Transcranial Doppler (TCD) techniques require application of the ultrasound to those areas of the skull where the bone is relatively thin. The frequency of the Doppler signal is also adjusted, and pulsed wave rather than continuous wave ultrasound is used to augment the transmission of ultrasound waves through the skull. Blood flow velocities from the cerebral arteries, carotid arteries, the basilar and the vertebral arteries can be sampled by altering the transducer location and angle, and the instrument's depth setting. The most common windows in the cranium are located in the orbit (of the eye), and in the temporal and suboccipital regions. Using TCD ultrasonography, cerebrovascular responsiveness to various physiological and pharmacological challenges can be assessed instantaneously, and various cerebral circulatory tests can be repeated frequently and safely. Rapid changes of cerebral perfusion over time can be easily followed, documented and analyzed and emboli and other blood flow irregularities can be detected with a high degree of sensitivity.

Emboli produce high intensity, transient Doppler ultrasound signals when they traverse sample volumes of a Doppler ultrasound instrument, and emboli may be detected directly as changes in Doppler signal amplitude. U.S. Pat. No. 5,348,015, for example, discloses methods and apparatus for ultrasonically detecting, counting and/or characterizing emboli in either arterial or venous circulation.

U.S. Pat. No. 6,196,972 relates to a pulse Doppler ultrasound system for monitoring blood flow including a graphical information display that simultaneously displays depth-mode and spectrogram data. The depth-mode display indicates various positions along the ultrasound beam axis at which blood flow is detected, with color indicating the direction of blood flow and varying intensity indicating the Doppler ultrasound signal amplitude or detected blood flow velocity.

Disturbances such as patient and probe movement and non-embolic debris in circulation reduce the sensitivity and accuracy of emboli detection using Doppler ultrasound techniques. Data processing techniques have been developed to increase the accuracy of Doppler ultrasound emboli detection methodologies. Several techniques are described in Wang et al, Emboli detection using the Doppler ultrasound technique, Technical Acoustics Vol. 22 No. 1E, pp. 15-18, 2003. U.S. Pat. No. 6,547,736 discloses a pulse Doppler ultrasound system for monitoring blood flow and detecting emboli in which subtraction of various background or artifact elements of the detected Doppler signals is provided to reduce false positive identifications of embolic events.

U.S. Pat. No. 6,616,611 discloses a Doppler ultrasound technique using clutter filtering to subtract out signals that may be intense but are low velocity and hence represent tissue rather than embolic events. A depth-mode display assists the user in determining whether a desired vessel has been located and a simultaneously displayed spectrogram is used for successfully and reliably locating and orienting the ultrasound probe and determining an appropriate sample volume depth.

One drawback of using acoustic techniques for measuring physiological parameters and detecting anomalies such as emboli using standard Doppler techniques is that localization of a desired CNS target area using an acoustic transducer is challenging and generally requires a trained, experienced sonographer to find and (acoustically) illuminate the desired target area, such as the middle cerebral artery (MCA). After locating the desired target area, the sonographer generally attaches a cumbersome and uncomfortable headset to the transducer that stabilizes the transducer position and reduces the effects of patient movement and other disturbances on the position of the transducer. The sonographer may be required to monitor acoustic readings and reposition or reorient the transducer intermittently to maintain the focus on the desired data acquisition area. This generally limits the use of Doppler ultrasound detection techniques to in-hospital and in-clinic situations where a trained sonographer is available.

There is increasing evidence that asymptomatic emboli are more frequent than clinical embolic events and are an important and detectable risk factor for transient ischemic attacks and stroke. TCD monitoring for asymptomatic cerebral emboli has been limited to relatively short recordings by equipment size and complexity and because probe fixation and operation typically requires a trained sonographer, as noted above.

Several systems for extended TCD monitoring have been proposed. U.S. Pat. No. 6,682,483 discloses methods and devices that provide three dimensional imaging of blood flow using long-term, unattended Doppler ultrasound techniques. Doppler ultrasound blood velocity data is collected in a three-dimensional region using a planar phased array of piezoelectric elements that lock onto and track points in the three-dimensional region that produce the locally maximum blood velocity signals. The automated tracking process may be used to provide a three-dimensional map of blood vessels and provide a display that can be used to select multiple points of interest for expanded data collection for long-term, continuous and unattended blood flow monitoring.

Long-term ambulatory monitoring for cerebral emboli using TCD using an ambulatory TCD system is described in Mackinnon et al., “Long-Term Ambulatory Monitoring for Cerebral Emboli Using Transcranial Doppler Ultrasound,” Stroke, 74-78, January 2004. The middle cerebral artery (MCA) Doppler signal was obtained via the transtemporal window with a conventional Doppler unit, with the ambulatory probe positioned at the transtemporal window. Both a proprietary elastic headband and glasses were initially evaluated as methods of probe fixation. The software monitored the Doppler signal quality and implemented an auto-search module that attempted to restore vessel insonation during recording when the signal dropped below a preset level. The search mode was activated at regular intervals to optimize insonation.

Spencer Technologies (Seattle, Wash.) has developed a TCD probe fixation system employing a headframe having a Doppler ultrasound probe mounted for contacting a subject's temporal region to access the temporal window for extended surgical monitoring, embolus detection monitoring and physiologic testing. The goal of the headframe is to prevent movement of the probe. The preferred methodology requires first locating and assessing the temporal window using a hand held ultrasound probe and then positioning and orienting the probe on the headframe at the desired temporal window location. It is recommended that the headframe be completely loosened or removed for 30-60 minutes every 3 hours of monitoring.

Deep vein thromboses in the peripheral vascular system, and particularly in the deep veins of the calves and thighs, produce narrowing of vessels that may interfere with circulation and may also embolize to produce embolic events in the heart, lungs, brain and other organs. Doppler ultrasound techniques are used to assess deep vein thromboses, but conventional techniques and devices do not provide long term monitoring, are not ambulatory, and suffer many of the disadvantages of Doppler ultrasound systems described above.

There is thus a significant need for methods and systems that provide long term, ambulatory monitoring of physiological parameters such as respiration, cardiac and/or blood flow parameters, events and anomalies and applicants' systems and methods are directed to addressing this need.

### SUMMARY OF THE INVENTION

The present invention provides ambulatory, noninvasive monitoring systems for acquiring and storing data relating to one or more of the following physiological parameters: respiration, heart rate, body temperature, electrical heart activity (electrocardiogram—ECG), blood flow velocity, blood pressure, intracranial pressure (“ICP”), presence of emboli to the brain and other parts of the body or other blood flow-related irregularities, such as stenoses or vasospasm, electrical brain activity (electroencephalogram—EEG), and blood oxygen composition or partial pressure (O₂, CO₂). Non-invasive pressure sensing devices such as electro-optical sensors, strain gauges and pressure transducers, for example, may be used to acquire data relating to respiration and heart rate, and conventional ECG techniques and electrodes may be used to acquire data relating to heart rate, blood oxygen composition, and electrical heart activity. Pulse oximetry techniques using, for example, electro-optical sensors, may be used to acquire data relating to heart rate and blood gas composition. Standard non-invasive blood pressure detection techniques using pressure cuffs or pressure transducers may be used to acquire data relating to blood pressure. EEG electrodes and data acquisition techniques are preferably: used to acquire data relating to brain activity. Non-invasive ultrasound techniques are preferably used to acquire data relating to blood flow properties, blood velocity, ICP, blood flow anomalies, the presence of emboli, and the like, and may also be used to acquire data relating to blood pressure. Movement detection devices may also be used to document the occurrence of motor seizures.

Monitoring systems of the present invention comprise one or more data acquisition devices such as one or more of the devices described above that, when placed in proximity to and/or in contact with a subject, acquires data relating to one or more of the desired parameters. Each of the data acquisition devices is in data transfer communication, via electrical leads or using a wireless data transfer protocol, with a patient data recording and storage device. The patient data recording and storage device has robust data storage capacity and may have data processing, analytical and display capabilities. Data recorded and stored is identified with a unique identifier corresponding to the individual subject for whom data is being acquired. Recorded and stored data is also identified with time and date information and a time and date display may be provided. A microphone and audio or mechanical recording activator may also be provided, enabling the subject to record observations, activities and events as desired. Patient initiated information may also be input into the patient data recording and storage device using patient selectable menu choices and other data input mechanisms.

In one embodiment, the patient data recording and storage device may be provided as a portable module designed for ambulatory subjects having an integrated power source and data transfer capabilities. Power sources that are rechargeable using electrically powered recharge devices are preferred. In another embodiment, the data recording and storage device may be provided as a typically stationary, table-top module designed for patients who have limited mobility, with power provided from external sources. The patient data recording and storage device preferably has data transfer capabilities that enable transfer of data from the storage device to a separate, data processing and analytical system, and/or to a larger capacity data archiving facility. Data transfer may be accomplished by physically removing a data storage subassembly from the data storage device, or using data transfer techniques employing a cable or a wireless protocol. Data transfer may be performed on a substantially real-time basis with substantially continuous or frequent transfers of data from the patient recording and storage device and/or data acquisition devices to a remote data processing and analytical system for substantially real-time monitoring. Alternatively, data transfer may be performed periodically and at intervals determined by the subject or professional caregiver or at data transfer intervals programmed into the device.

The patient data recording and storage device may be operated to collect and/or store data continuously or intermittently and may optionally have analytical and/or display capabilities as well. In one embodiment, manual activation and shut-off mechanisms are provided, enabling a subject to activate and inactivate the data acquisition devices and record and store data. In another embodiment, one or more data acquisition routines is programmed into the patient data recording and storage device and desired data acquisition routines may be selectable by the subject or pre-set by a health care professional. Data acquisition routines may involve, for example, acquiring data from one or more data acquisition devices at certain time intervals or during certain physiological states, acquiring data for certain time intervals, and transmitting and storing the data in specified databases or in one or more storage location(s).

The system may be programmed or programmable to compare real-time, acquired data with predetermined or programmable standards and identify anomalies. Alarm and/or notification triggers may be preset or programmable at predetermined limits and alarms and notifications may be delivered locally, to the subject, or remotely to a monitoring service or health care provider. Certain data acquisition and analysis functions and capabilities may be selected and programmed by health care professionals and certain functions and capabilities may be programmable or selectable by users. The ambulatory devices may be provided with individual identifiers and may have data transmit-receive capabilities that enable acquired data to be transmitted to a remote data storage and/or analysis system, and that enable control systems, data acquisition and analysis routines, limits, and the like to be transmitted from a remote location to the ambulatory device. Data may be transferred from an ambulatory

Ambulatory devices may also have localization capabilities incorporating VHF, GPS, satellite and/or triangulation location systems. These systems are capable of notifying care-givers or services having a companion receiver, in real time, of anomalies in a subject's physiology, location or the like, thus allowing the monitoring entity to take action to find and assist the subject. The inventive system may thus function as a rapid alarm, providing identification of the subject, the location of the subject and an indication of the problem the subject is having. The system may be applied, for example, to children, hikers, at-risk persons with known medical conditions, and ambulatory, as well as bed-ridden, patients.

A separate data processing and analytical system generally provides data retrieval and sophisticated data analysis when desired by a health care professional and incorporates or is used in conjunction with a display system for presenting visual representations of the analyzed data. Substantial efficiencies are achieved because a single analytical system may be located remotely from the subject being monitored and used to evaluate patient data for a relatively large patient population. This analytical system is used by doctors and other health care professionals to evaluate the condition of a patient and formulate diagnoses, prognoses, etc. Subject data may also be transferred, from the patient data recording and storage device and/or from the separate data processing and analytical system to a remote data storage and archiving facility.

A standard cardiac monitor with event capability provides continuous recording of respiration, heart rate and event-triggered ECG. The measurements are compared periodically to a calibrated norm and recording of the ECG data is activated for the duration of an event or for a predetermined time period when acquired measurements deviate from the norm by a predetermined amount. This device may be used by athletes, runners, cyclists, trekkers, climbers, patients undergoing cardiac rehabilitation and subjects at risk for or evidencing symptoms of cardiac irregularities. A calculation of the amount of calories lost during a measurement or exercise period may be performed and displayed and a body temperature reading may be measured and displayed as well. The inclusion of a location identifying technology such as GPS and wireless communication capability enables this system to also serve as an alarm and provide speedy location of the subject. A beacon function may be included to facilitate this safety-related use where wireless operation is not possible.

Systems of the present invention may be employed as a highly effective child and infant monitor. Such a monitoring device may incorporate many of the functions identified above. The child's respiration may be continuously monitored and any meaningful deviation from a predetermined or empirically determined standard may trigger an audible alarm both at the data acquisition device and at the matched receiver device. This type of child monitoring device may additionally incorporate heart rate and/or ECG monitoring capability that may be automatically activated and monitored or that may be activatable by a companion receiver/controller device. This system may be set up so that a parent or supervisor may monitor location and communicate (two-way) with the child at any time by remote. In the event of anyone tampering with the child, the child could push an alarm button activating the alarm to the parent and turning on the VHF transmitter and/or GPS and microphone. This would also occur automatically if anyone tried to tamper with or remove child's monitoring system. An on-site alarm and beacon may be incorporated for added safety.

Systems of the present invention that monitor respiration and/or heart rate and/or ECG may also be used for detection of sleep apnea without requiring a subject to stay at a specialized laboratory or wear uncomfortable breathing monitors. The system described herein allows detection of apnea and other abnormalities in a subject's own home, at a low cost, and can be used to monitor the success of any therapy instituted. The system may also detect respiratory depression in infants and children, and can therefore be used to detect and prevent SIDS by monitoring the breathing status of children during sleep.

Another aspect of methods and systems of the present invention relates to monitoring devices that, in addition or alternatively to having one or more cardiac monitoring functions, have the capacity to acquire data relating to blood and blood flow parameters using non-invasive techniques and similarly analyze, report, trigger alarms, and provide effective long term and remote monitoring of blood flow conditions and anomalies. Systems of the present invention incorporating a noninvasive ultrasound detection device are useful for providing long term monitoring of circulation, blood pressure and blood flow velocities, ICP, and for detecting blood and blood vessel anomalies such as stenoses, vasospasm and emboli.

In one embodiment, a “long term” emboli detection trace corresponding to data acquired over a time period of at least several hours and up to several days or months is provided to illustrate trends and fluctuations in emboli over time that may be predictive of risk for pulmonary embolism, stroke, transient ischemic attacks, and the like. These systems are based on Doppler or other acoustic measurements, such as acoustic scatter, taken from a target site on or within or in proximity to a blood vessel such as the MCA, a carotid artery, another cranial blood vessel or, for peripheral blood monitoring applications, a peripheral blood vessel. Monitoring systems incorporating ultrasound data acquisition devices preferably incorporate an automated target vessel locating and focusing feature that scans a tissue volume and identifies and focuses on blood vessel(s) and blood vessel volume(s) exhibiting desired acoustic properties relating to desired blood flow characteristics. This automated target vessel locating and focusing feature preferably updates and adjusts the focus and/or orientation of one or more acoustic data acquisition devices at regular intervals during long term monitoring operations.

Blood flow and blood flow anomaly detection and monitoring is preferably accomplished using an ambulatory ultrasound source/receiver system that may be mounted on or applied to a patient's skull, neck, leg, trunk or the like, where it preferably locates and maintains focus on a desired vessel with little or no assistance from an operator. An initial environmental assessment may be made, if desired, to assess the characteristics of the environment between the acoustic source and the target vessel site, and calibration or programming of the data acquisition device for use with a particular blood vessel may be facilitated by a health care professional. The initial environmental assessment may be determinative of various method and system parameters. Environmental assessments may additionally be updated at intervals throughout a diagnostic or monitoring procedure.

A property of blood flow, such as acoustic scatter or flow velocity, may be determined in any blood vessel. For determination of ICP and emboli detection applications, arteries that traverse, or enter or exit CNS tissue (collectively, “cranial blood vessels”) are preferred. Peripheral veins in the leg or thigh are preferred for detection of emboli that are predictive of risk for pulmonary embolism. Blood flow properties are preferably detected using ultrasound techniques such as Doppler and Transcranial Doppler (TCD) ultrasound techniques, which are well known in the art.

Doppler ultrasound techniques may be used to acquire data relating to blood flow velocity and ICP and may be used, as well, to detect stenoses, vasospasm, emboli and other blood flow anomalies. In addition or alternatively, acoustic properties of tissue, including blood, blood vessel walls, tissue in proximity to blood flow, and other tissue sites, may be assessed, for example, by collecting acoustic scatter data using an ultrasound transducer aimed at or having a focus on a blood vessel, and/or at another target site. For purposes of detecting emboli, the target vessel site is preferably a cranial blood vessel or a blood vessel that leads to or traverses the brain, or a peripheral blood vessel such as a deep vein in an extremity. Cranial blood vessels may be accessed by contacting an ultrasound transducer to the temporal window through the skull or by contacting an ultrasound transducer to a location on the neck or upper chest where acoustic access to a cranial blood vessel such as a carotid artery is available.

Monitoring of at least one of the common carotid arteries, cervical internal carotid arteries, middle cerebral arteries, subclavian arteries, vertebral arteries and basilar arteries is preferred for cerebral blood flow monitoring and emboli detection. In one preferred system, monitoring of a carotid artery that traverses the neck is provided using a portable ultrasound transducer mounted on an elastic band attachable around a subject's neck. Systems of the present invention incorporating emboli detection features may be used to assess a subject's risk for stroke and other blood flow abnormalities and to assess the efficacy of treatment regimen. Monitoring of a deep venous vessel in the peripheral vascular system, such as deep veins in the legs, is preferred for peripheral blood flow monitoring and emboli detection and may be used to assess a patient's risk for pulmonary embolism and other blood flow abnormalities, as well as assess the efficacy of a treatment regimen.

Methods and systems of the present invention provide spatial location of desired target areas based on their acoustic properties and automated focusing of an acoustic source at one or more desired target area(s). Multiple target vessels or multiple target locations within multiple vessels or multiple locations within a single vessel may be monitored simultaneously or sequentially using ultrasound data acquisition techniques. Suitable source/detector combinations and transducer assemblies for scanning and locating desired target areas are described.

Blood flow monitoring and emboli detection methods and systems that monitor a carotid artery, for example, may operate in one or more modes. A carotid artery monitoring regimen may involve acoustically illuminating (scanning) a relatively large tissue volume and analyzing received acoustic signals from a relatively large tissue volume to identify the location of the artery within a larger region of tissue. Thereafter, a focused acoustic beam may be aimed to acoustically illuminate substantially an entire cross-section of the artery, or one or more focused acoustic beams may be aimed simultaneously or sequentially to illuminate distinct smaller volumes within the cross-section of the artery. Acoustic detection patterns may match the transmit patterns or may differ from the transmit patterns. A multi-frequency acoustic array may be used in conjunction with multi-frequency transmit and detection schemes to provide enhanced detection of desired events and conditions, such as the presence of emboli.

Systems of the present invention provide long term monitoring of ambulatory patients to identify events and abnormalities that are asymptomatic and/or infrequently experienced and also provide effective assessment of treatment regimen. They are suitable for use with ambulatory subjects and may also be used in non-ambulatory applications such as in hospital rooms, surgical suites, ambulances, nursing and other long term care facilities, and the like. Integrated monitoring systems, for example, may be employed to provide comprehensive patient monitoring within a hospital or institution at a fraction of the cost of conventional monitoring equipment. At present, hospitals have only a fraction of their beds monitored, and the only monitoring systems are cardiac monitoring devices that require operation by trained nurses. A very small percentage of cardiac arrest patients in-hospital survive, due to the very critical few minutes before the code team gets to them. Alarm and notification systems of the present invention alert nurses or other care-givers in a residential or hospital facility, or monitoring professionals in a remote monitoring facility and expedite the delivery of essential and appropriate care and intervention. Methods and systems of the present invention can be used to notify medical staff at the very early moments of a respiratory or cardiac arrest or of a major embolic event or blood flow abnormality, thereby greatly increasing the chances of a successful outcome.

### BRIEF DESCRIPTION OF THE FIGURES

FIG. 1 illustrates a schematic diagram showing various ambulatory components of systems of the present invention.

FIG. 2 is a schematic flow diagram depicting data acquisition, processing and communication functions of systems of the present invention.

FIG. 3 is a schematic diagram illustrating one embodiment of a patient data recording and storage device.

FIG. 4 illustrates the major cerebral vessels, including the middle cerebral artery (MCA), the target of standard transcranial Doppler procedures, and schematically illustrates an acoustic source emitting acoustic interrogation signals in a scanning mode.

FIGS. 5A and 5B show, schematically, the use of a transducer array of the present invention in a scanning mode (FIG. 5A) used to locate the target area of interest based on its acoustic properties, and in a focusing and data acquisition mode (FIG. 5B).

FIGS. 6A and 6B illustrate an exemplary patient interface unit having an acoustic source/detector combination of the present invention.

### DETAILED DESCRIPTION OF THE INVENTION

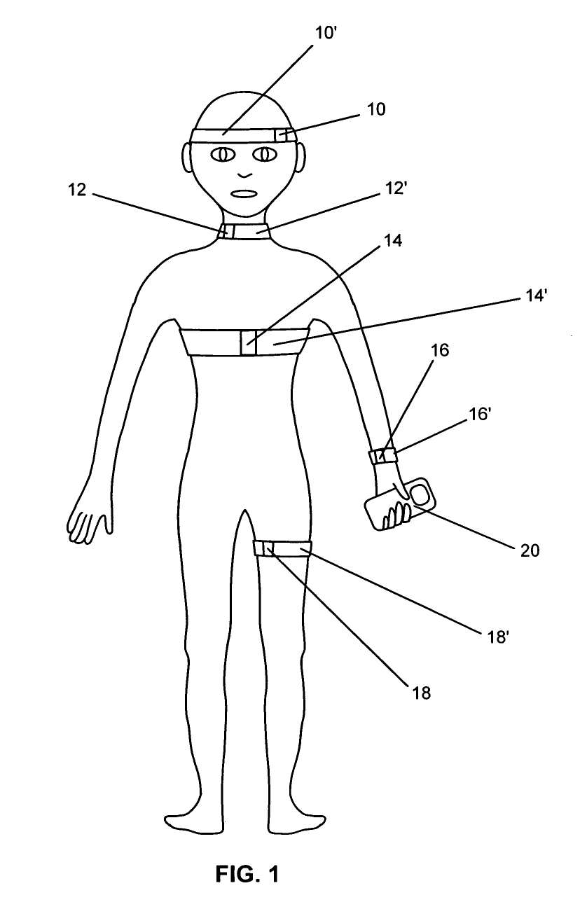

Methods and systems of the present invention may comprise numerous combinations of features and capabilities, as described herein. As illustrated schematically in FIG. 1, a system of the present invention comprises one or more noninvasive data acquisition devices 10, 12, 14, 16, 18 or a similar device provided in proximity to or in contact with a patient's skin or outer surface. In one embodiment, data acquisition devices 10, 12, 14, 16 and 18 are mounted or incorporated in or integrated with flexible, elastomeric bands 10′, 12′, 14′, 16′, 18′ or alternative mounting systems sized to fit snugly around one or more features of a patient's anatomy. One or more of the bands may be adjustable to facilitate snug fitting of the band and contact or close proximity of the data acquisition device with a surface of the subject. The data acquisition devices may be provided at a fixed position on the respective band, or they may be movable and adjustable on the respective bands to facilitate positioning of the device at desired locations. In another embodiment, not illustrated, one or more data acquisition devices may be provided in connection with a garment or another form-fitting assembly.

In the embodiment illustrated schematically in FIG. 1, data acquisition device 10 is intended for mounting on a subject's skull in proximity to a temporal window, data acquisition device 12 is intended for mounting on a subject's neck for data acquisition from blood vessels traversing the neck, such as a carotid artery, and data acquisition device 18 is intended for mounting on a subject's extremity, such as a thigh, for data acquisition from blood vessels such as deep veins, traversing the extremity. Each of these data acquisition devices preferably comprises an ultrasound transducer or transducer array capable of insonating and scanning a tissue target site to identify a target vessel of interest, focusing on one or more desired volume(s) of the vessel of interest, and acquiring acoustic data from the vessel of interest that relates to blood pressure, blood flow velocity and/or blood flow anomalies such as emboli.

Data acquisition device 14, intended for mounting on a subject's chest, preferably comprises one or more pressure sensing devices such as a pressure transducer or strain gauge for detection of respiration and measurement of heart rate and/or one or more electrodes for acquisition of ECG signals. Pressure sensing devices for acquisition of respiration and heart rate data may alternatively or additionally be mounted on another portion of the subject's trunk or provided in a data acquisition device 16 intended for mounting on a subject's arm.

In one embodiment of the system of the present invention an elastic, pressure-sensing material such as KINOTEX® or another type of polymer foam consisting of a layer of thin cellular elastomers of urethane or silicon that electro-optically measures continuous mechanical respiration and/or heartbeat, is implemented as a data acquisition device. The polymer foam may be provided in the form of an elastic band or a close-fitting garment and may include an inner and/or outer skin of cotton or other comfortable material providing a patient contacting or wear surface. One or more ECG sensors and/or leads may be used in conjunction or integrated with a pressure sensing band or garment for acquisition of data relating to respiration, heart rate and ECG from the same wearable, ambulatory device.

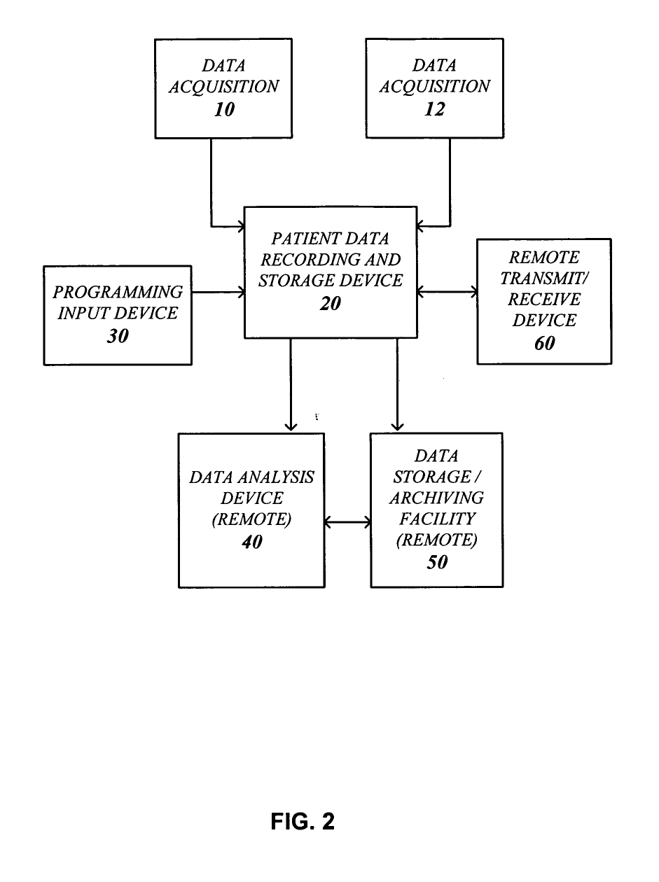

As shown schematically in FIG. 2, each of the data acquisition devices 10, 12, 14, 16, 18 or the like, is in data flow communication with a data recording and storage device 20. Data may be acquired in one or more of the acquisition devices on a substantially continuous basis or intermittently, and is conveyed to a data recording and storage device wirelessly or via electrical leads. Alternatively, data acquisition may be initiated or terminated by the user or a health care professional. Data acquisition times and patterns may be programmed or programmable via the data recording and storage device 20 and/or via another external programming input controller.

Data recording and storage device 20 may, in addition to data recording and storage capabilities, have data analysis capabilities provided, for example, in software or firmware. High capacity data recording and storage may be provided in a variety of formats, such as Smart Media Cards, Flash Cards, in embedded Flash caches or other types of embedded digital storage media and may be provided as a removable data storage medium or as an embedded medium. For ambulatory applications, data recording and storage device 20 is preferably a relatively small, portable, battery operated device that can be easily carried by the user in a pocket or bag, worn on a user's belt, placed in proximity to the subject, or the like. Data acquired from data recording and storage device 20 is preferably marked with a unique identifier assigned to the patient using the device.

Data processing and analysis capabilities provided in recording and storage device 20 may be programmed or programmable. In one embodiment, data acquired may be processed in device 20 to determine heart rate, respiratory rate, body temperature, calories burned, or the like, for example, which may be displayed continuously or intermittently on a device display. Acquired data may be averaged over programmed or programmable time periods and otherwise processed according to methods that are well known in the art. Data recording and storage device 20 may also be programmed or programmable by the end-user or a medical professional using selectable embedded programs and limits or using an auxiliary programming input device 30. Device 20 may be programmed or programmable to incorporate threshold limits or value ranges and data processing routines activating an alarm or notification, locally or remotely, when acquired data exceeds a programmed limit or falls outside a predetermined range.

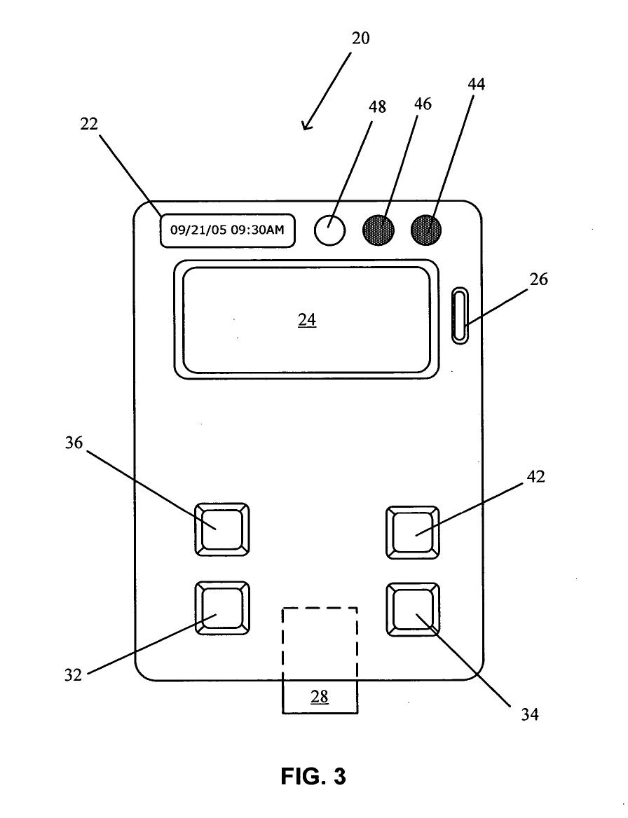

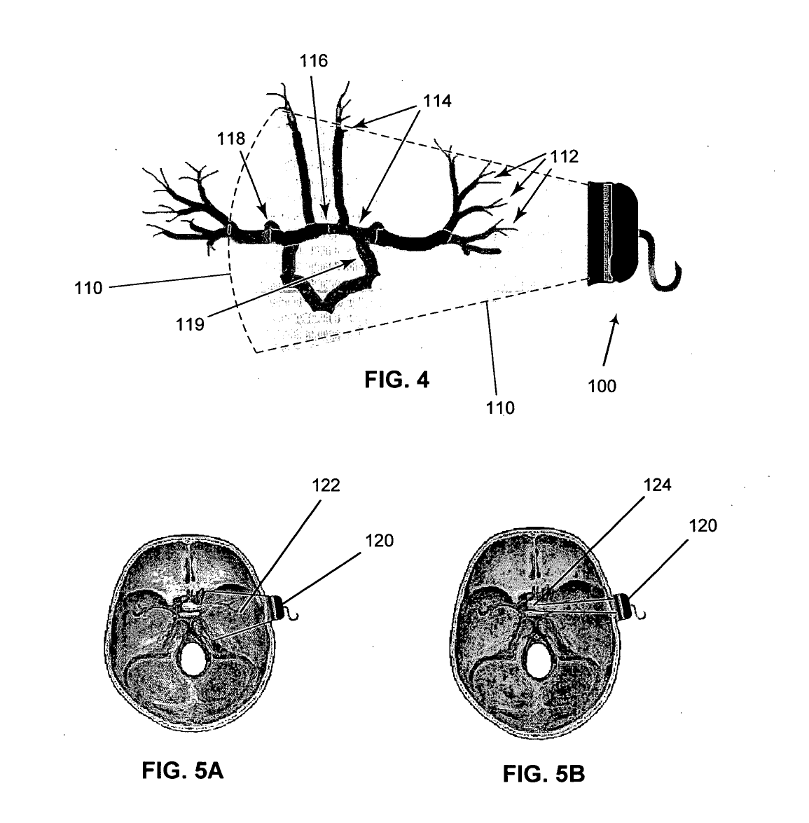

Data acquisition and storage device 20 is illustrated schematically in FIGS. 1 and 3 as a portable, ambulatory device, but it will be recognized that the data acquisition and storage device that interfaces with the patient data acquisition devices may alternatively be provided as a stationary, table-top type system designed for use in hospital and residential care facilities. A stationary system may have enhanced data processing and display functions compared to the ambulatory device and may provide longer term storage capabilities and enhanced alarm and notification functions.

Data stored in device 20 is preferably transferable to a separate analytical device 40 for more sophisticated data processing, analysis, patient diagnosis, and the like. Analytical device 40 may be installed, for example, at a health care or monitoring facility and operated by health care professionals. Data may be transferred by removing a removable data storage medium and physically transferring the stored data to analytical device 40, or data may be transmitted using wireless or wired techniques from device 20 to a remote analytical device 40 for data processing and analysis. Data processing, analysis and monitoring services may thus be centralized and receive and analyze data from numerous data acquisition and storage devices used by numerous patients.

Data stored in device 20 and/or data and analytical information generated by analytical device 40 is preferably transferable to a data storage or archiving facility 50 that is separate and optionally remote from data storage device 20 and analytical device 40. When a separate data storage or archiving facility 50 is used, data is preferably transferable between archiving facility 50 and data analysis device 40 upon command. Device 20 may also have transmit/receive capability to analytical device 40 for relaying alarms or notifications, for example, or to an independent matched transmit/receive device 60. VHF, GPS, satellite and triangulation location methodologies may be implemented.

FIG. 3 illustrates a highly schematic diagram illustrating one embodiment of a data recording and storage device 20. Device 20 incorporates a time/date display 22, and a data display 24 for displaying cardiac and/or blood flow parameters calculated using data acquired from the data acquisition devices. Data relating to one or more of respiration rate, body temperature, heart rate, blood oxygen content, calories burned, blood flow velocity, ICP and blood pressure may be displayed, for example, for viewing by the user. Alarms and notifications may also be displayed. A display actuator 26 is preferably provided for manually activating and inactivating the display. Data storage capability may be incorporated as an integral part of device 20, or one or more insertable and removable data storage subassemblies 28 may be provided for data storage. High capacity data storage capabilities are preferred.

Data recording and storage device 20 may additionally incorporate a manual data recording activator mechanism 32 that may be activated by a user, for example, upon a user's perception of symptoms or unusual conditions, to record and store data during and/or prior to an activation period. A data recording and storage inactivator mechanism 34 may also be provided to permit the user to manually terminate data recording and storage upon return to perceived normal physiological conditions. A data input/download function 36 may also be provided to allow the user or a medical professional to input data or information or to download programming or analytical data processing capabilities to the data recording and storage device 20. A voice recording actuator 42 may be provided, allowing a user or medical professional to record voice or auditory input to device 20 through microphone 44. Audible alarms or notifications may be provided through amplifier 46 and visual alarms and notifications may be provided through visual alarm 48. It will be appreciated that many modifications to device 20 as it is illustrated in FIG. 3 may be made to provide the various features described herein and to deliver relevant data in a fashion that is most useful to both the subject and a medical professional.

In one embodiment, a system of the present invention may incorporate one or more ultrasound transducer or transducer array data acquisition devices mounted in a patient affixation device such as a headset or an elastic band suitable for mounting on a subject's skull, neck or extremity. Acoustic data is used, in this embodiment, to detect and monitor blood flow parameters such as blood flow velocity, changes in blood flow and blood flow parameters, arterial blood pressure, ICP, and blood flow anomalies such as emboli and the like. All of these blood-related parameters may be detected using techniques that are known in the art and the device may be programmed or programmable to activate one or more alarms or notifications when the data acquired is outside predetermined limits or ranges. Data indicative of blood flow velocity and ICP may also be acquired and analyzed to provide real-time data relating to values for blood flow velocity and ICP and changes in blood flow velocity and ICP, which may be clinically useful parameters. Similarly, acoustic data is used to detect and monitor for the passage of emboli

Ultrasound sources and detectors may be employed in a transmission mode, or in a variety of reflection or scatter modes, including modes that examine the transference of pressure waves into shear waves, and vice versa. Detection techniques involving measurement of values for or changes in acoustic scatter, such as back scatter or forward scatter, or reflection, and particularly backscatter, are preferred for use in many embodiments of methods and systems of the present invention. Exemplary acoustic data that may be used to determine blood flow parameters and anomalies according to the present invention include: values for or changes in acoustic scatter, including values of and changes in the amplitude, phase and/or frequency of acoustic signals, values for or changes in length of scattered signals relative to the interrogation signal, values for or changes in the primary and/or other maxima and/or minima amplitudes of an acoustic signal within a cardiac and/or respiratory cycle; values for or changes in ratios of the maximum and/or minimum amplitude to that of the mean or variance or distribution of subsequent signals within a cardiac cycle, values for or changes in temporal or spatial variance of scattered or emitted signals at different times in the same target location and/or at the same time in different target locations, values for or changes in endogenous and/or induced brain tissue displacement or relaxation, and rates of change for such displacements, such as the velocity or acceleration of displacement, and the like, and combinations of these data.

Multiple acoustic interrogation signals may be employed, at the same or different frequencies, pulse lengths, pulse repetition frequencies, intensities, and the multiple interrogation signals may be emitted from the same location, or multiple locations, simultaneously and/or sequentially. Acoustic scatter data may be collected, for example, from a blood vessel at different points along the vessel, within or outside the cranial cavity, or from multiple sites at or in proximity to different vessels. Scatter from single or multiple interrogation signals may be detected at single or at multiple frequencies, at single or multiple time points, and at single or multiple locations. In one embodiment, methods and systems of the present invention may be used to localize blood flow abnormalities and anomalies within different tissue samples, thereby localizing areas of trauma or dysfunction.

In one embodiment, Doppler techniques are used to measure flow velocity and to detect blood flow anomalies such as emboli in a desired blood vessel, such as the MCA (V_mca), a carotid artery, or a peripheral vein. Doppler is a preferred ultrasound technique and can provide substantially continuous measurement of flow velocity. Many types of Doppler devices are known in the art and the Spencer Technologies TCD 100M Power M-Mode Digital Transcranial Doppler device is one such device that is suitable for collecting acoustic data from cranial blood vessels.

In addition to blood flow velocity in one or more selected vessel(s), acoustic data may also be acquired and processed to provide real-time determination of blood pressure, particularly arterial blood pressure (ABP). ABP may be determined using acquired acoustic data and techniques described in PCT International Publication No. WO 02/43564, which is incorporated by reference herein in its entirety. ICP may also be determined, in real time and during long term monitoring, using acoustic data acquired as described herein. Several methods and systems for determining ICP are described, for example, in PCT International Publication No. WO 2004/107963 A2, which is incorporated by reference herein in its entirety.

If ABP, ICP, blood flow velocity and flow anomaly data are acquired in an integrated data acquisition device such as an ultrasound transducer array as described herein, the data is conveniently synchronous with respect to acquisition time, substantially reducing or eliminating the need for data synchronization. In other embodiments, ABP, flow velocity and flow anomaly data may be acquired using different devices and/or synchronization rates, with the data being collected and processed in an integrated processing unit that provides data synchronization as necessary. ABP may also be monitored non-invasively, for example, using a conventional arm or leg cuff or another non-invasive device, such as the VASOTRAC® device manufactured by Medwave, Inc., 4382 Round Lake Road West, St. Paul, Minn. 55112-3923. Blood vessel and/or blood flow characteristics and ABP may be measured on a substantially continuous or an intermittent basis using acoustic data.

Various data processing techniques may be used to condition acquired acoustic data. These include, for example, downsampling and/or resampling of telemetry and Doppler flow data to provide that each linear signal record occupies the same amount of space so that standard signal processing techniques may be employed more easily. Data cleaning may also be implemented to ensure that all signal records are continuous, within expected physiologic ranges, and appropriate for further processing. Anomalies may trigger an alarm or notification to provide monitoring information and alert the user or a monitoring professional that a blood flow anomaly has occurred or that the data acquisition device is no longer operating properly. Phase alignment of cardiac cycle boundaries is generally implemented to ensure the input data is in phase with regard to cardiac cycle boundaries.

If pulse-domain transformation is performed, the data may require alignment, such as through cross-correlation spectrum analysis or other methodologies. Transformation of the linear, phase-aligned, time-domain telemetry and Doppler flow records to two-dimensional, normalized pulse-domain records may be desirable. This is a multi-step process and may involve calculation and storage of beat-to-beat instantaneous heart rate, normalization of each cardiac cycle to a fixed number of samples, and moving pulse-window smoothing or envelope calculation for the V_mca Doppler flow data. Systems of the present invention for monitoring blood flow parameter and blood flow anomaly events preferably provide trend analysis and data display features. One suitable output display provides: (1) one or more trace(s) of embolic events over a “long term” period of time of at least several minutes and up to several hours or days to illustrate trends in patient embolic activity; (2) a trace of “instantaneous” or “short term” flow anomalies, determined over several cardiac cycles; and (3) additional graphical representations that may aid in guidance of an acoustic transducer or transducer array, as described below.

A calibration step using a measure of blood pressure taken with a conventional blood pressure device may be incorporated in a system having the capability of making blood pressure determinations using acoustic data. Acoustic proxies for the pulsatility of the blood vessel—such as oscillation rate of the blood vessel wall—may be substituted for direct measures of those quantities. In this method, the spontaneous changes in the diameter (or other geometric property) of the vessel being monitored are assessed using ultrasound, and this information is related (e.g., using correlation techniques) to synchronous Doppler flow measurements within the same vessel. Since the diameter (or other geometric property) of the vessel is a function of the pressure being exerted against the wall of the vessel by blood, and since the velocity of blood flow is dependent on the diameter (or radius) of the vessel through which the blood travels, blood pressure can be calculated from flow velocity measured by Doppler. By simultaneously measuring the pulsatility of the blood vessel of interest and the Doppler flow velocity proximal and distal to this site, continuous blood pressure can be determined.

One aspect of the present invention relates to the use of acoustic source/detector assemblies for acquiring data relating to blood flow parameters and for detecting blood flow anomalies. In operation, an acoustic source/detector combination, such as a Doppler source/detector, is stably mounted, or held, in proximity to a patient's body surface, such that the focus of the acoustic source(s) is adjustable to provide an acoustic focal point on a blood vessel or other target site within the patient's body. For CNS target sites, the acoustic source/detector is stably mounted, or held, in proximity to a cranial window, such that the focus of the acoustic source(s) is adjustable to provide an acoustic focal point on a cranial blood vessel. For vessel target sites such as the carotid arter(ies), the acoustic source/detector is stably mounted on a surface of the neck to provide an acoustic focal point on and/or within the vessel(s) of interest. Similarly, for peripheral target sites, the acoustic source/detector is stably mounted on a surface of the extremity, such as on the thigh, to provide an acoustic focal point on and/or with the peripheral vessel(s) of interest.

The acoustic source/detector combination is preferably provided as a unitary component, but separate acoustic source and detector components may also be used. The acoustic source/detector combination may be provided in connection with a mounting structure or accessory that provides temporary adherence to desired patient sampling locations and may be provided as a single use component.

Various types of acoustic transducers and acoustic transducer arrays may be used as acoustic source/detector assemblies and acoustic data acquisition components of the present invention. A single acoustic transducer, or a singer acoustic transducer array may be operated both as a source and a detector, or separate source and detector transducers or transducer arrays may be provided. Conventional PZT acoustic transducers may be implemented as acoustic data acquisition components in methods and systems of the present invention. Acoustic transducer arrays composed of cMUT and PVDF cells or elements may also be used and are preferred for many implementations. PZT, cMUT and PVDF acoustic transducers and arrays may be combined in various data acquisition components and operated in acoustic source and/or receiver modes in yet other embodiments.

In one embodiment, the acoustic source/detector combination may be mounted on a stabilizer, or on or in a structure, such as a helmet-type structure or headband or neckband or legband that may be mounted on the patient at a location providing acoustic access to the desired blood vessel. An applicator containing an acoustically transmissive material, such as an acoustic gel, may be placed between the surface of the acoustic source/detector combination and the patient's skin. Steering of the acoustic device may be accomplished manually or using automated mechanisms, such as mechanical or electronic steering mechanisms. Such mechanisms are well known in the art.

Methods and systems of the present invention incorporate systems and methods for locating and acoustically illuminating and/or probing a desired target area in a reliable and automated fashion, without requiring a trained sonographer. FIG. 5 illustrates major cerebral vessels, including the middle cerebral artery (MCA), the target of standard transcranial Doppler procedures and a target for acoustic measurements used in the methodology employed for detecting blood flow parameters and anomalies described above. The anterior cerebral arteries 114, anterior communicating artery 116, internal carotid artery 118 and posterior communicating artery 19 are shown. The darkened blood vessel branches denote blood flow towards acoustic device 100, while cross-hatched blood vessel sections denote flow away from the transducer. An acoustic source/detector assembly 100 useful in methods and systems of the present invention is schematically illustrated to the right of the cerebral vessels. Acoustic source/detector assembly 100 emits acoustic interrogation signals in a wide beam 110 in a scanning mode as described below, in which a relatively large target area is acoustically illuminated prior to the focusing and localization of acoustic signals on one or more smaller target site(s).

Thus, another aspect of the present invention relates to methods and systems for locating and acoustically illuminating and/or probing a desired target site in an automated fashion using an array comprising a plurality of acoustic source and/or detector elements. An acoustic transducer/receiver array may be employed in a scanning mode, for example, to acquire acoustic data from numerous sites within a larger target area. Based on the acoustic data collected in the scanning mode, localized sites within the target area may be selected as target sites for focused acoustic illumination and/or probing. Localized target sites may be selected, or predetermined, based on any aspect of the acoustic data collected in the scanning mode, such as acoustic scatter amplitude, phase and/or frequency maxima or minima, tissue stiffness properties, endogenous and/or induced tissue displacement properties, rates of change of such properties, and the like.

Focusing of elements of an acoustic transducer/receiver array on selected target sites may be accomplished in an automated fashion using mechanical or electronic beam steering and other automated acoustic focusing methodologies. In another embodiment, an automated system is provided that locates a desired target site within a larger target area in a scanning mode, focuses on the desired target site for acquisition of acoustic data, and thereafter periodically scans the target area and repositions the acoustic focus, if necessary, to maintain the focus of the acoustic source at the desired target site. Multiple target sites may also be located in a scanning mode and focused on sequentially and/or simultaneously for acoustic data acquisition from multiple target sites using acoustic transducer/receiver array assemblies of the present invention. Systems incorporating suitable arrays of acoustic source and/or detector elements are disclosed.

FIG. 5A illustrates, schematically, the use of a scanning acoustic transducer assembly 120 of the present invention that acoustically illuminates and acquires acoustic data from multiple points within a broad target area 122, such as a large portion of the cerebral blood vessel complex, in a scanning mode. Based on the acoustic data acquired in the scanning mode, localized target sites 124 within the scanned area may be identified and elements of the transducer assembly are focused on localized target site(s) for acquisition of acoustic data from the desired target site(s), as shown in FIG. 5B. Selection of localized target site(s) may be predetermined based on various acoustic properties, including the amplitude (or any amplitude derivative) of acoustic scatter data, Doppler analysis of acoustic scatter data, phase or frequency of acoustic data, changes in the primary and/or other maxima and/or minima amplitude, phase or frequency of acoustic signals within a cardiac and/or respiratory cycle or other period, or determinations derived from acoustic data, such as flow velocity, tissue stiffness properties, endogenous and/or induced tissue displacement properties, acoustic emissions associated with such displacements, rates of change of such properties, and the like.

For monitoring blood flow parameters and anomalies using methods of the present invention, the selection of a desired localized target site, such as the MCA, a carotid artery, a peripheral vein, or another blood vessel, is preferably accomplished by scanning the desired target area, as shown in FIG. 5A, and determining the localized site of highest amplitude acoustic scatter, or highest Doppler or flow velocity values, which represents the vessel of interest. Acoustic elements of the acoustic source/receiver data acquisition component may then be focused on one or more localized blood vessel sites for acoustic data acquisition. Other sites having unique acoustic properties may also be located. Coordinates for target vessel volume location and values for acoustic properties may be recorded and stored, over time, and displayed in a variety of formats.

Various noninvasive, non-acoustic detection modalities may be employed alternatively or additionally to locate internal physiological structures, including blood vessels such as the MCA, prior to acquisition of acoustic data. Near infra-red spectroscopy (NIRS), magnetic resonance, and other techniques are known and used, for example, to image and locate internal physiological structures. Such techniques may be used in association with the methods and systems of the present invention for locating internal physiological structures prior to assessment of acoustic properties.

Using methodologies and assemblies described below, an acoustic source/detector combination, preferably an acoustic transducer array comprising multiple transducer elements, is operable in both a scanning mode and a focusing mode. One or more acoustic source element(s) of the acoustic data acquisition component acoustically illuminates a relatively broad desired target area in a scanning mode to identify target sites having predetermined or desired acoustic properties, thus identifying the target site(s) as blood vessel(s). When the acoustic source has identified one or more target sites having the predetermined or desired acoustic properties, one or more of the acoustic source(s) may be manually or automatically focused on the desired target site(s) for operation in an acoustic interrogation or data acquisition mode. The acoustic source may also be programmed to monitor acquired acoustic data and to adjust the positioning and/or focus of the source to maintain the focus of selected or predetermined acoustic source(s) on the desired target site. Similarly, acoustic source(s) may be programmed to acquire data from a plurality of predetermined or programmed target sites at predetermined time points.

Having identified the location of the target vessel in a scanning mode, one or more target vessel volumes may be selected for data acquisition and analysis. For methods and systems involving data acquisition from the MCA, as described above, the acoustic focus and data acquisition volume generally represents substantially the entire cross-section of the target MCA vessel. For methods and systems involving data acquisition from a carotid artery or a peripheral vessel, it may similarly be desirable to acquire acoustic data in a volume that represents substantially the entire cross-section of the target carotid or peripheral vessel. In some embodiments, the focus and beam size of the acoustic source(s) may substantially match the focus and beam size of the acoustic detector(s), so that acoustic data is acquired from substantially the entire vessel volume that was acoustically illuminated.

For blood vessels having a relatively large cross-sectional volume, such as the carotid arteries and peripheral veins, for example, multiple sample volumes that are volumetrically smaller than a sample containing the entire vessel cross-sectional volume may be monitored simultaneously and/or sequentially. In a relatively large vessel such as a carotid artery or peripheral vessel, for example, it is desirable for some applications to acquire data from one or more relatively small vessel volumes at or near the center of the vessel and from one or more relatively small vessel volumes at or near the periphery of the vessel. Data from numerous vessel volumes may be acquired simultaneously or sequentially. The focus and beam size of an acoustic source may be substantially larger than that of one or more acoustic detectors to acoustically illuminate a relatively large vessel volume and provide data collection from one or more smaller vessel volumes within the larger acoustically interrogated volume. Alternatively, the vessel volume interrogated may substantially match the vessel volume from which acoustic data is acquired.

Monitoring of blood vessels such as a carotid artery may be accomplished using a generally higher frequency array than may be used for emboli detection, for example, in the MCA. Acoustic frequencies of from about 0.5 MHz to 15 MHz, more preferably from about 1.0-10 MHz, may be used for carotid artery monitoring to provide high resolution acoustic data with a generally low level of artifacts. Vessel monitoring may also be accomplished using multiple frequencies for acoustically interrogating and/or for acoustic data acquisition over time and/or over vessel sample volumes to facilitate enhanced detection of blood flow parameters and anomalies. Acoustic transducer source and detector elements of the present invention may, in fact, be programmed to collect one or more types of acoustic data from a single or multiple target sites, at one or more frequencies and at one or more times. Acquisition of acoustic data, using methods and systems of the present invention, is preferably accomplished in an automated fashion.

Methodologies for scanning and locating desired target areas based on their acoustic properties may be based on “range-Doppler” search methodologies that were originally developed, for example, for programming torpedoes to hunt targets such as submarines. Range-Doppler processing is an efficient implementation of matched filtering that has been used in the radar and sonar signal processing community for many years. It is a robust technique, in part because it makes very few assumptions about the statistical nature of the environment and targets that it encounters. Range-Doppler processing provides a useful decomposition of the spatial and temporal (i.e. Doppler) scattering properties of the target of interest. Sensor time series data are divided into frames, often overlapped, multiplied by the transmitted waveform replica and then transformed into the frequency domain via the Fast Fourier Transform (FFT) algorithm. These operations implement, very efficiently, a bank of matched filters, each matched to a narrow range of Doppler shifts. Range-Doppler processing affords separation of targets in terms of their range and speed relative to the acoustic device. Intracranially, MCA flow is by far the largest target, which makes it a natural for this ‘search and home in’ approach.

Other methodologies for finding and maintaining an acoustic focus on a desired target area are also applicable. Acoustic holography techniques such as those described in Porter, R. P., P. D. Mourad, and A. Al-Kurd (1992), Wavefront reconstruction in variable, multimode waveguides. J. Opt. Soc. Am., A9(11) 1984-1990 and Mourad, P. D., D. Rouseff, R. P. Porter, and A. Al-Kurd (1992), Source localization using a reference wave to correct for oceanic variability, J. Acoust. Soc. Am., 92(1) 1031-1039, may also be used. Using acoustic holography techniques, signals from a target are combined in a convolution with signals from a reference source after each is measured on an acoustic array. The net result is a formula whose maximum occurs at the target site. To determine ICP using acoustic holography techniques, for example, all of the acoustic fields may be replaced by the Fourier transform of the acoustic field, or a component of the Fourier transform of the acoustic field, e.g. the Doppler signal. In this embodiment, the Fourier transform of the acoustic backscatter from an acoustic array serves as the target signal, and the forward scatter from a TCD or array placed on the opposite temple may be used as the reference source. These signals would be mathematically combined to find and maintain an acoustic focus on a desired target area.

In another embodiment, it would be useful to have the option for the user to have the opportunity to assist the automated targeting, user independent aspects of the present invention. This may be useful, for example, for cases where systems for automatically identifying the feature of interest may not be uniquely converging on that feature, or so that the user can validate whether or not the feature chosen by the computer is, in their opinion, the optimal feature. The key idea is that the feature of interest will be known to represent a local if not global minimum or maximum among a spatial distribution of values of the feature of interest. We will use the example of finding the maximum flow velocity in the middle cerebral artery, where the velocity in the middle cerebral artery is known to have a range of values spatially distributed along the middle cerebral artery, with the understanding that this technology is not limited to this application.

An exemplary acoustic system providing an automated targeting feature while allowing user participation in targeting may utilize conventional TCD systems made by DWL, Spencer Technologies, Nicolet, or the like, where the acoustic sensor consists of a single transducer element, and where the acoustic system provides information only along the beam of the single transducer for a given orientation of that transducer. Here, the user manually manipulates the transducer so that it insonifies different portions of the cerebral architecture, and electronically steers the depth along the transducer beam axis. The user would be guided by the real-time display of information, along with the user's memory of what the display has shown in the preceding moments, to seek out the maximum in flow velocity in the desired vessel. One portion of the display may provide the real time value of the variable of interest at a position relative to the face of the transducer (reported in absolute units, or arbitrary units, since the actual depth is not important) that is chosen by the user with a cursor designated for this purpose. The display may provide, for example, the real time value of flow velocity in the MCA, otherwise known as the spectrogram of the flow.

Another portion of the display may provide a graphical image designed to communicate to the user, at any given orientation of the transducer, the direction of larger values of flow in the MCA relative to the real time position of the cursor. This may take the form of two arrows pointing in different directions, e.g. one pointing ‘up’ one pointing ‘down,’ where up and down are known to the user to represent deeper relative to the present position of the cursor, and more shallow relative to the present position of the cursor, respectively. If there are local maxima in flow velocity in both directions, the direction in which a greater maximum exists would be designated by having a brighter arrow pointing in that direction. These flow velocity gradients may be calculated within the associated controller component by measuring the Doppler shift along all of the points insonified at a given moment by the transducer to provide a real-time calculation of the local gradient of the flow velocity. This calculation may be performed using a variety of well-known mathematical formulae (one-sided differences, centered differences to a variety of orders, etc). The absolute position of the local flow velocity maximum in flow in the MCA need not be known or reported or displayed to the user.

What the user gains from this analysis is a direction, relative to the current position of the cursor, which position need not be defined, of the local maximum in flow velocity. The user may then manipulate the cursor to report the spectrogram at a deeper or a shallower position along the acoustic beam and judge for themselves whether they have achieved a local maximum in flow velocity. By providing guided exploration of the flow velocity along the beam axis in this fashion, in combination with physical manipulation of the relative position or angle of the transducer, the user will be able to locate the flow velocity maximum in a guided fashion.

Standard TCD devices also allow for the device to emit sound whose amplitude is tied to the flow velocity at a given point along the beam of the transducer, the one, in particular, whose spectrogram is shown to the user. Such supplemental information would be of interest to the user of the present invention. In addition, one could designate the intensity of the display to increase or decrease as the absolute value of the flow velocity increased or decreased as the cursor was manipulated along the beam of the transducer. In this way visual information would supplement the aural information already available to the user.

Using an acoustic array comprising a relatively dense distribution of acoustic transducers rather than a single transducer or a sparse array, one may have, at any given moment, information relating to the relative spatial distribution of flow velocity or other blood parameters in depth at a variety of angles from the center of the acoustic beam. A user assist feature may provide a display showing the direction of the local flow velocity maximum. Using a transducer array, however, locational information relating to the direction of maximum flow velocity may be provided in additional dimensions, and the user may be guided by an arrow pointing in each of the three possible directions of cursor movement relative to the real time cursor position. One set of arrows may indicate the local maximum is deeper than, or shallower than, the present cursor position. Another set of arrows may indicate that the local maximum is more anterior or posterior to the present cursor position. Yet another set of arrows may indicate that the local maximum in flow velocity is more superior or inferior to the present cursor position. This information may be calculated as described above, using Doppler analysis of acoustic backscatter from the field of positions insonified by the transducer array. The user's positioning of the array may be guided by this information, along with supplemental aural and visual information as described above, including the instantaneous spectrogram at the position of interest, to move the cursor, and re-examine the spectrogram.

Acoustic systems and transducer assemblies for locating and illuminating one or more desired target site(s) on or within a blood vessel are described below. The acoustic methods and systems described below may be useful for any application in which collecting data relating to an acoustic property of a desired target site is required. Acoustic transducer arrays of the present invention are generally thin and generally comprise a single layer or thickness of transducer elements. Stacked, multiple layer transducer cells, or elements, may be used for some applications. The transducer elements or cells may be arranged on a single plane to form a generally flat, planar array, or they may be arranged to form a curved or a geometrically stepped array.

Transducer arrays having various configurations and structures may be useful for applications contemplated in this disclosure. For applications involving monitoring of a carotid artery, a rectangular array having more cells aligned in one direction than in the other is generally preferred to facilitate monitoring of a vessel volume along a length of the carotid artery. For monitoring applications involving monitoring of multiple vessel volumes simultaneously or sequentially, fewer cells may be employed in a transmit mode to acoustically illuminate a generally broad target area and more cells may be employed in a receive mode to acquire acoustic data from a plurality of different vessel volumes.

In one embodiment, data acquisition components comprising acoustic source/detector combinations of the present invention comprise a plurality of capacitive micromachined ultrasound transducer (cMUT) cells. cMUT ultrasound transducers are manufactured using semiconductor processing techniques and have sufficient power and sensitivity to transmit and receive at diagnostic ultrasound energy levels, which is necessary and sufficient for purposes of the present invention. The transducer elements are fabricated using small capacitive diaphragm structures mounted on a silicon substrate. cMUT transducer arrays have the potential of being produced very inexpensively, and may also have the support electronics integrated onto the same chip.

cMUT ultrasound transducer cells comprise a positive electrode, generally provided as the top electrode and a negative electrode, generally provided as the bottom electrode. The top electrode is generally provided on or in connection with a flexible membrane and the bottom electrode is generally provided on or in connection with a substrate, such as a silicon substrate. Insulating supports are provided to form a sealed chamber between the positive and negative electrodes. The chamber may contain a gas or liquid or gel-like substance, or it may be provided as an evacuated chamber. The diaphragm structures of the cMUT ultrasound transducer convert acoustic vibrations into a modulated capacitance signal, or vice versa. A DC bias voltage is applied and an AC signal is either imposed on the DC signal in transmission or measured in reception. In general, cMUT transducer elements may be operated in various modes of transmit and receive operation, including unbiased mode, non-collapsed mode, collapsed mode and collapsed snapback mode (transmit only). One advantage of using cMUT transducer cells, elements and arrays is that the electronics may be provided on or in the cell structure, greatly simplifying the electronic communication to and from the array and facilitating programmable array features.

A cMUT transducer array is composed of multiple individual cMUT ultrasound transducer cell structures arrayed as elements, with the elements arrayed in rows and/or columns and/or smaller divisions forming the array. The number of cMUT transducer cells forming each transducer element, and the number of elements forming an array may be varied, depending on the array application. cMUT transducer arrays having various configurations may be assembled and used in the present invention. cMUT transducer arrays can be configured and operated to achieve acoustic transmission and sensitivity levels sufficient to perform as acoustic transmit/receive devices suitable for use in medical devices, such as TCD devices. More specifically, cMUT transducer arrays having a plurality of cMUT element columns operated at an 80V bias, 28 Vac to transmit acoustic energy to CNS target sites at intensities of up to 1.75 W/cm², while typical transmission intensities of only about 0.6-0.7 w/cm² are required for determining cerebral blood flow using conventional TCD acoustic devices. cMUT transducer arrays operated experimentally at an 80Vbias and at a gain of 60 and 80 dB to receive signals from CNS target sites in a range of less than 4 to greater than 6 cm from the array at a level sufficient to make Doppler determinations. cMUT transducer cells and elements may be arranged in different combinations to provide cMUT transducer arrays having different capabilities. If each of the cMUT cells is provided with independently controlled or controllably electronics, each of the cMUT cells may act as a transducer element and an array may be provided as a plurality of independently controlled or independently controllable cMUT cells. More typically, a transducer element comprises a plurality of cMUT cells that is electronically controlled or controllable as a unit. Thus, each of the elements composed of multiple cMUT transducer cells are controlled or controllable as a unit. Alternatively, a plurality of the elements, such as elements forming a row or a column, may be electronically controlled or controllable as a unit to provide a cMUT transducer array comprising a plurality of row or column transducer elements. A one-dimensional (1D) array may be composed of a single transducer element comprising multiple cells, while a two-dimensional (2D) array is composed of multiple transducer elements arranged in a generally planar, two-dimensional configuration.

In one embodiment, two cMUT acoustic arrays, each composed of a single or multiple transducer elements, are aligned in a “Mills Cross” configuration in which two transducer arrays are arranged generally orthogonal to one another, which allows one array to sweep vertically in send and receive modes and the other to sweep horizontally in receive and send modes. In this implementation, a first linear cMUT transmit array may be steerable in a first direction, such as a vertical direction and a second linear cMUT receive array is arranged generally orthogonal to the first linear array and may be steerable in a direction orthogonal to the first direction. The two, crossed linear cMUT arrays alternatively transmit and receive ultrasound beams while steering the sending and listening beams, to identify and focus on acoustic signals having the desired property.

In another embodiment, an acoustic array comprising PVDF (polyvinylidene fluoride) film transducers is used as an acoustic detector array, alone or in combination with a cMUT array or a single element PZT transducer employed as the source. In an exemplary embodiment comprising a PVDF array in combination with another transducer or array, the source transducer or array transmits sound through the PVDF array, sweeping the sound in a single dimension generally perpendicular to the arrangement of the PVDF array. The PVDF array serves as the acoustic detector, receiving and processing acoustic signals.

Acoustic transducer arrays suitable for use in systems of the present invention may alternatively comprise a combination PVDF/cMUT array(s). The combined depth of the arrays is generally quite small and may be on the order of about 1 cm. A cMUT array may be arranged below a PVDF array, for example, with the PVDF array arranged closest to the subject's surface during use. In this configuration, the cMUT array is operated as the acoustic source and transmits acoustic beams through the PVDF array. The cMUT array may be composed of a 1D or 2D array comprising one or more cMUT acoustic elements. The PVDF array may also be provided as a 1D array or as a 2D array. When acoustic source(s) and/or detector(s) are provided as 2D arrays, they are capable of sending and/or detecting acoustic signals in two dimensions, rather than a single direction.

Acoustic arrays suitable for use in systems of the present invention may also comprise one or more combination(s) of PVDF array(s) and PZT transducer(s). A cMUT array may similarly be used in combination with a PZT transducer. The PVT transducer is generally mounted below the PVDF or cMUT array and transmits as an acoustic source through the PVDF or cMUT array in a single, broad beam. In these embodiments, the PZT transducer generally serves as the acoustic source and the PVDF or cMUT array generally serves as the acoustic detector. Each of the aligned transducer elements in the cMUT array is controlled or controllable as a unit.

One of the advantages of employing ultrasound transducer array components as described above in systems of the present invention is that multifunctional arrays may be provided in a relatively high power, yet inexpensive system. Such arrays are very versatile, are capable of performing multiple acoustic functions and may be pre-programmed or programmable to provide desired functions, and may be provided as disposable or single-use elements of an integrated clinical diagnostic system. In one embodiment, acoustic arrays of the present invention are provided as a single-use acoustic data acquisition component of a medical device, such as a blood flow monitoring system, comprising one or more acoustic transducer arrays in operative communication with a controller component having data processing, storage and/or display capability. The one or more acoustic transducer arrays may communicate with the controller component by means of one or more detachable cables, or using a radio frequency, infrared or other wireless technology. The transducer array(s) may be steerable and may be programmed to scan one or more target areas having certain boundaries or parameters, and locate one or more desired target site(s) based on preselected or selectable acoustic properties. The transducer array(s) may furthermore be programmed and/or controllable to establish and maintain a focus by directing ultrasound beams having a preselected intensity, amplitude, phase, frequency, etc., to the target site(s) in an automated fashion. Transducer arrays of the present invention may also be programmed to collect acoustic data from multiple target sites simultaneously, or at different times. In one embodiment, a transducer array, or a plurality of arrays, may be programmed to operate alternatively as acoustic sources and detectors. In one embodiment, multiple transducer arrays used for monitoring multiple patients; provide data to and communicate with a single data processing, storage and display device.

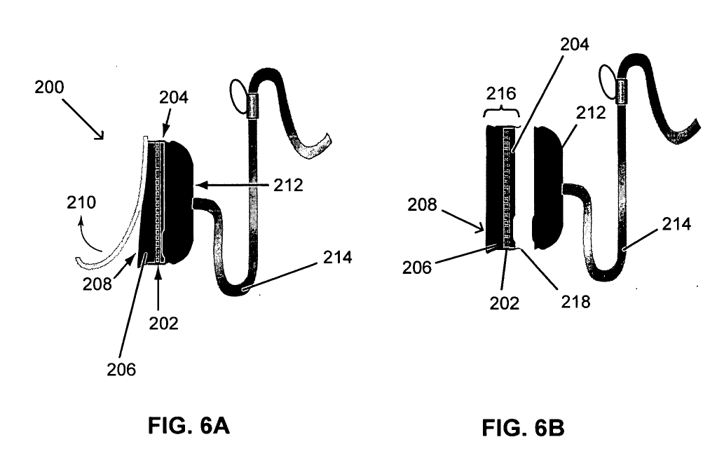

FIGS. 6A and 6B illustrate one exemplary embodiment of acoustic data acquisition components comprising acoustic source/detector systems, such as acoustic arrays, of the present invention. In the embodiments illustrated in FIGS. 6A and 6B, both disposable and non-disposable elements are shown. In this system of FIG. 6B, costly elements of the acoustic system are provided as non-disposable components, while less costly components, which require close interaction with a patient and, perhaps, sterilization, are provided as a single-use component.

FIG. 6A illustrates an acoustic data acquisition component 200 comprising an acoustic transducer array 202 that interfaces with an array electronics component 204 and an acoustic transmission component 206 that facilitates high fidelity acoustic transmission between transducer array 202 and a subject's body surface. Acoustic transmission component 206 preferably comprises a sealed enclosure containing an acoustically transmissive material, such as an acoustic gel having uniform properties and being substantially free from acoustically significant discontinuities, such as bubbles. Acoustic transmission component 206 may incorporate an adhesive substance on a least a portion of an exposed surface 208 to facilitate temporary adherence of the data acquisition component to a subject's body surface. Exposed surface 208 bearing an adhesive substance may be protected by a detachable cover 210 that may be removed prior to placement on a subject's body surface.

The transducer array and array electronics component may be permanently mounted in or on a structure 212 that facilitates communication of data and/or power to and/or from a controller component. Structure 212 may incorporate control and/or power features or may provide operable connection of the transducer array and array electronics to control and/or power features that are housed in a separate controller component. Data acquisition component 200 may communicate with a controller component through a structure 212 and cable 214, as illustrated in FIG. 6A, or communication may be provided using alternative communications methodologies, such as RF or other wireless communications systems. If transducer array 202 and array electronics component 204 are mounted permanently or semi-permanently in structure 212, acoustic transmission component 206 may be provided as a single use component and may be affixed to an exposed surface of transducer array 102 prior to mounting on a subject's body surface.

Alternatively, acoustic transducer array 202, array electronics component 204 and acoustic transmission component 206 may be provided as a single use acoustic data acquisition component 216, as illustrated schematically in FIG. 6B. Single use acoustic data acquisition component 216 has an electronics interface component, illustrated schematically as wire 218, that provides communication between array 202 and array electronics component 204 and electronics and/or power capabilities provided in structure 212 or in a remote controller component. The electronics interface component provided in connection with data acquisition component 216 may be a hard-wired interface component that relies on contact with a mating interface component in structure 212, or it may be provided as a wireless interface communications component. In this embodiment, single use data acquisition components 216 may be packaged in a sterile or non-sterile fashion.

In this embodiment, an acoustic array is provided as part of a single use or disposable system element, in combination with a patient interface component. The acoustic array is preferably in contact with acoustically transmissive material, such as an acoustic gel, that provides high fidelity acoustic transmission into and from the target area. The acoustically transmissive material is preferably interfaced with a contact material, such as an adhesive material, that facilitates temporary positioning and affixation of the disposable system element to a patient's skin. The patient contact material may be protected by a removable cover, which is removable at the time of use. The disposable system element, including the acoustic array, may be provided as a unitary element that may be sterilized and packaged for one-time use.

Alternative single use systems and elements may also be employed. In one such alternative system, acoustically transmissive material layers may be provided as a separately sterilized, packaged component that is designed to interface with a non-disposable component including the acoustic array(s). Such layers may be provided with an adhesive layer on one side for contact with the patient's skin. Or, a recess may be provided for manual application of acoustically transmissive material. It will be evident that many different embodiments and arrangements of disposable and non-disposable elements may be employed.

This compact, disposable array element may be placed in contact with the temple of the patient and, when activated, electronically scans a target area of interest, such as the area of cerebral blood vessels, and then focuses the acoustic source(s) and detector(s) on the target site of interest, such as the MCA, the carotid artery, or a peripheral vein. The acoustic array monitors and stays focused on the target area of interest during operation. In this embodiment, the acoustic array forms part of a disposable assembly including an acoustic gel, or another acoustic material that facilitates transmission of acoustic signals at the interface with the patient's skin during operation. The exposed surface of the acoustic gel is preferably interfaced with one or more adhesive elements that facilitate temporary placement on and consistent contact with a desired patient surface. A removable cover may be provided over the acoustic gel to preserve the acoustic array and other components.

These elements may be provided as a disposable unit, as shown in FIG. 6B, that is mountable on non-disposable elements of the system. Non-disposable elements of the system may include mounting hardware, one or more cables or wireless transmission interfaces, and a data processing, storage and display device (not shown).

Placement of the acoustic source(s) and detector(s) on a subject for assessment of acoustic properties of a target blood vessel may be at known “acoustic windows” in the cranium for detection of blood flow parameters and anomalies in cranial vessels such as the MCA. Placement of the acoustic source(s) and detector(s) for assessment of acoustic properties and detection of blood flow parameters and/or anomalies of a carotid vessel is preferably on the neck or upper chest of a subject. Placement of the acoustic source(s) and detector(s) for assessment of acoustic properties and detection of blood flow parameters and/or anomalies of a peripheral vein is preferably on the thigh or calf of a subject. The placement of the source(s) with respect to the detector(s) will depend on the acoustic data desired—e.g., for collection of back scatter acoustic data, the source(s) and detector(s) are in proximity to one another, while the source(s) and detector(s) are positioned generally opposite one another for collection of forward scatter acoustic data. Acoustic scatter or reflection data may be collected at various angles by placing the source(s) and detector(s) at various locations on the patient.

Methods and systems of the present invention may be used in a variety of settings, including emergency medicine settings such as ambulances, emergency rooms, intensive care units, and the like, surgical settings, in-patient and out-patient care settings, residences, airplanes, trains, ships, public places, and the like. The techniques used are non-invasive and do not irreversibly damage the target tissue. They may thus be used as frequently as required without producing undesired side effects. The methods and systems of the present invention do not require patient participation, and patients that are incapacitated may also take advantage of these systems. The methods and systems for assessing tissue properties, including ICP, may be used on a continuous or intermittent basis for monitoring tissue properties or ICP.

All of the publications described herein, including patent and non-patent publications, are hereby incorporated herein by reference in their entireties.

We claim:

1. A system for long term monitoring of least one of the following physiological parameters: respiration, heart rate, body temperature, electrical heart activity, blood flow velocity, blood pressure, intracranial pressure (ICP), presence of emboli to the brain and other parts of the body or other blood flow-related irregularities, such as stenoses or vasospasm, electrical brain activity, and blood oxygen composition or partial pressure (O₂, CO₂) comprising at least one noninvasive data acquisition device for acquiring data relating to at least one of the physiological parameters communicating with at least one data recording and storage device for recording and storing data relating to one of the physiological parameters.

2. A method for long term monitoring of embolic events in the bloodstream comprising acoustically scanning a tissue volume including a carotid artery and acquiring scanning data relating to the acoustic properties of the tissue volume, selecting at least one desired target carotid artery vessel site based on the scanning data, insonifying the at least one desired target carotid artery vessel site and acquiring acoustic data from the at least one insonified target carotid artery vessel site, and recording, storing and monitoring the acquired acoustic data to identify embolic events.
